(19) United States
(12) Patent Application Publication
(10) Pub. No.: US 2008/0045424 A1
(43) Pub. Date: Feb. 21, 2008

(54) Additive for Lubricating Oil and Fuel Oil, Lubricating Oil Composition, and Fuel Oil Composition

(75) Inventors: Nobuaki Shimizu, Chiba (JP); Tadashi Katafuchi, Chiba (JP)

Correspondence Address: OBLON, SPIVAK, MCCLELLAND MAIER & NEUSTADT, P.C., 1940 DUKE STREET, ALEXANDRIA, VA, 22314, US

(73) Assignee: IDEMITSU KOSAN CO., LTD., TOKYO (JP)

(21) Appl. No.: 11/573,092

(22) PCT Filed: Aug. 4, 2005
(86) PCT No.: PCT/JP05/14342

§ 371 Date: Feb. 2, 2007

(30) Foreign Application Priority Data
Aug. 4, 2004 (JP) 2004-227702

(51) Int. Cl. C10M 135/26; C07C 69/34; C10L 1/18

(52) U.S. Cl. 508/509; 44/385; 560/129

(57) ABSTRACT

An additive for lubricating oils and fuel oils comprising a disulfide compound, as the main component, represented by the general formula (I)

R¹OOC—CR³R⁴—CR⁵(COOR²)—S—S—CR¹⁰(COOR⁷)—CR⁸R⁹—COOR⁶ (I)

wherein R¹, R², R⁶, and R⁷ each independently represent a hydrocarbyl group having from 1 to 30 carbon atoms and may contain an oxygen, sulfur, or nitrogen atom; and R³ to R⁵ and R⁸ to R¹⁰ each independently represent a hydrogen atom or hydrocarbyl group having from 1 to 5 carbon atoms, a lubricant oil composition and fuel oil composition comprising the additive are prepared to provide a sulfur-based extreme-pressure additive which exhibits excellent load carrying capacity and wear resistance and corrosion resistance against nonferrous metals and can be used in lubricating oils and fuel oils, and to provide a lubricating oil composition and fuel oil composition containing the additive.

### TECHNICAL FIELD

The present invention relates to additives for lubricating oils and fuel oils, lubricating oil compositions, and fuel oil compositions. More particularly, the present invention relates to an additive for lubricating oils and fuel oils which comprising a disulfide compound, as the main component, having a specific structure and to a lubricating oil composition and fuel oil composition containing the additive, the additive exhibiting excellent properties suitable for friction modifiers, and particularly extreme pressure additives and antiwear agents.

### BACKGROUND ART

Lubricating oils have been used in driving units, such as internal combustion engines, automatic transmissions, shock absorbers, and power steerings (systems), to smooth their operations it is known that lubricated surfaces are worn by friction and eventually seized under high-output and high-load conditions due to insufficient lubricating ability. Thus lubricating oils containing extreme pressure additives and anti-wear agents are used.

Unfortunately, conventional extreme pressure additives are not always satisfactory because of insufficient resistance against seizure, metal corrosion, or wear due to interaction with other additives.

Metalworking oils used for metal working such as cutting, grinding, and deformation processing are prepared by compounding mineral oils and synthetic hydrocarbon oils with oiliness agents such as alcohols, fatty acid esters, and fatty acids and extreme pressure additives, as an attempt to improve workability.

Further improvements in workability of such metalworking oils are required in view of higher productivity and energy saving. In addition, conventional chlorinated extreme pressure additives, which have been widely used, cause deterioration of working environment, such as rashes of operators and rust of metal works. Thus, the use of the chlorinated extreme pressure additives trends to be suppressed.

As metalworking oils satisfying these requirements, oil solutions composed of base oils, sulfurized olefins containing active sulfur atoms, and perbasic sulfonates are commercially available.

The above commercially available metalworking oils exhibit high welding resistance and can prevent abnormal wear of tools by chipping, and ripping on worked surfaces. In working with repeated friction cycles at relatively low load, however, active sulfur atoms promote corrosion wear of tools. This causes an increase in frequency of change or grinding of the tools, resulting in decreasing production efficiency. On the contrary, the production efficiency often decreases in metal working without abnormal wear.

Hydraulic oils are power transmission fluids used for power transmission, control, and buffering in hydraulic systems such as hydraulic equipment, and function as lubrication of sliding portions.

Essential properties of the hydraulic oils are high resistance against load seizure and wear. These properties are imparted by compounding base oils such as mineral oils and synthetic oils with extreme pressure additives and antiwear agents. Although conventional extreme pressure additives exhibit satisfactory resistance against load seizure, these are not sufficient in wear resistance and corrosion resistance.

Urging requirements for gear oils and particularly vehicle gear oils are improvements in wear resistance and oxidation resistance due to recent severer driving conditions, such as heavier load and long-distance transportation accompanied by development of highway networks, and prolonged periods of oil change.

Lubricating base oils have been primarily compounded with extreme pressure additives and antiwear agents such as sulfurized oil and fat, sulfurized olefin, phosphoric and thiophosphoric acid compounds, and zinc dithiophosphate. However, further improvements in wear resistance and oxidation resistance and a reduction in ratio of wear coefficient (low speed to high speed) are required.

It is known that lubricating ability of fuel oils decreases as the degree of hydrogenation increases and that fuel pumps using highly refined fuel are readily worn. Thus, fuel for recent high-performance turbines requires high lubricating ability, and additives for fuel oils should have excellent properties, namely, improved lubricating ability and reduced wear resistance caused by extreme-pressure layers formed by adsorption of additives on metal surfaces of fuel-system parts or component parts.

Sulfur-based extreme pressure additives have been regularly used for extreme pressure additives of lubricating oils. The sulfur-based extreme pressure additives having sulfur atoms in their molecules are dissolved or homogeneously dispersed in base oils, and exhibit extreme-pressure effects. Examples of known additives include sulfurized oil and fat, sulfurized fatty acid, sulfurized esters polysulfides, sulfurized olefin, thiocarbamates, thioterpenes, and dialkyldithiopropionates. These sulfur-based extreme pressure additives, however, are not always satisfactory because these cause corrosion of metal and exhibit insufficient seizure resistance or wear resistance due to interaction with other additives.

### DISCLOSURE OF INVENTION

Under such a circumstance, an object of the present invention is to provide a sulfur-based extreme-pressure additive used for lubricating oils and fuel oils exhibiting superior load-carrying capacity and wear resistance, and low corrosion attack against nonferrous metals compared to conventional sulfur-based additives and to provide a lubricating oil composition and fuel oil composition containing the additive.

The inventor has discovered that an additive for lubricating oils and fuel oils comprising a disulfide compound having a specific structure as the main component fits the object after extensive study. The present invention has been accomplished under such a finding.

The present invention provides:
(1) An additive for lubricating oils comprising a disulfide compound, as the main component, represented by the general formula (I):

R¹OOC—CR³R⁴—CR⁵(COOR²)—S—S—CR¹⁰(COOR⁷)—CR⁸R⁹—COOR⁶ (I)

wherein R¹, R², R⁶, and R⁷ each independently represent a hydrocarbyl group having from 1 to 30 carbon atoms and may contain an oxygen, sulfur, or nitrogen atom; and R³ to R⁵ and R⁸ to R¹⁰ each independently represent a hydrogen atom or hydrocarbyl group having from 1 to 5 carbon atoms;
(2) An additive for lubricating oils comprising a disulfide compound, as the main component prepared by oxidative coupling of a mercaptoalkanedicarboxylic acid diester represented by the general formula (II) and/or general formula (III):

R¹OOC—CR³R⁴—CR⁵(COOR²)—SH (II)

wherein R¹ and R² represent a hydrocarbyl group having from 1 to 30 carbon atoms and may contain an oxygen, sulfur, or nitrogen atom; and R³ to R⁵ each independently represent a hydrogen atom or hydrocarbyl group having from 1 to 5 carbon atoms:

R⁶OOC—CR⁸R⁹—CR¹⁰(COOR⁷)—SH (III)

wherein R⁶ and R⁷ represent a hydrocarbyl group having from 1 to 30 carbon atoms and may contain an oxygen, sulfur, or nitrogen atom; and R⁸ to R¹⁰ each independently represent a hydrogen atom or hydrocarbyl group having from 1 to 5 carbon atoms;
(3) An additive for lubricating oils comprising a disulfide compound, as the main component, prepared by oxidative coupling of a mercaptoalkanedicarboxylic acid represented by the general formula (IV) and/or general formula (V).

HOOC—CR³R⁴—CR⁵(COOH)—SH (IV)

wherein R³ to R⁵ each independently represent a hydrogen atom or a hydrocarbyl group having from 1 to 5 carbon atoms:

HOOC—CR⁸R⁹—CR¹⁰(COOH)—SH (V)

wherein R⁸ to R¹⁰ each independently represent a hydrogen atom or a hydrocarbyl group having from 1 to 5 carbon atoms; and then esterifying the product with an alcohol represented by the general formula (VI):

R¹¹—OH (VI)

wherein R¹¹ represents a hydrocarbyl group having from 1 to 30 carbon atoms and may contain an oxygen, sulfur, or nitrogen atom;
(4) An additive for fuel oils comprising a disulfide compound, as the main component, represented by the general formula (I)

R¹OOC—CR³R⁴—CR⁵(COOR²)—S—S—CR¹⁰(COOR⁷)—CR⁸R⁹—COOR⁶ (I)

wherein R¹, R², R⁶ and R⁷ each independently represent a hydrocarbyl group having from 1 to 30 carbon atoms and may contain an oxygen, sulfur, or nitrogen atom; and R³ to R⁵ and R⁸ to R¹⁰ each independently represent a hydrogen atom or hydrocarbyl group having from 1 to 5 carbon atoms;
(5) An additive for fuel oils comprising a disulfide compound, as the main component, prepared by oxidative coupling of a mercaptoalkanedicarboxylic acid diester represented by the general formula (II) and/or general formula (III):

R¹OOC—CR³R⁴—CR⁵(COOR²)—SH (II)

wherein R¹ and R² represent a hydrocarbyl group having from 1 to 30 carbon atoms and may contain an oxygen, sulfur, or nitrogen atom; and R³ to R⁵ each independently represent a hydrogen atom or hydrocarbyl group having from 1 to 5 carbon atoms:

R⁶OOC—CR⁸R⁹—CR¹⁰(COOR⁷)—SH (III)

wherein R⁶ and R⁷ represent a hydrocarbyl group having from 1 to 30 carbon atoms and may contain an oxygen, sulfur, or nitrogen atom; and R⁸ to R¹⁰ each independently represent a hydrogen atom or hydrocarbyl group having from 1 to 5 carbon atoms;
(6) An additive for fuel oils comprising a disulfide compound, as the main component, prepared by oxidative coupling of a mercaptoalkanedicarboxylic acid represented by the general formula (IV) and/or general formula (V):

HOOC—CR³R⁴—CR⁵(COOH)—SH (IV)

wherein R³ to R⁵ each independently represent a hydrogen atom or a hydrocarbyl group having from 1 to 5 carbon atoms:

HOOC—CR⁸R⁹—CR¹⁰(COOH)—SH (V)

wherein R⁸ to R¹⁰ each independently represent a hydrogen atom or a hydrocarbyl group having from 1 to 5 carbon atoms; and then esterifying the product with an alcohol represented by the general formula (VI):

R¹¹—OH (VI)

wherein R¹¹ represents a hydrocarbyl group having from 1 to 30 carbon atoms and may contain an oxygen, sulfur, or nitrogen atom:
(7) A lubricating oil composition comprising (A) a lubricating base oil and (B) the additive for lubricating oils as claimed in any one of Aspects (1) to (3);
(8) A lubricating oil composition as claimed in Aspect (7) wherein the content of the component (B) is 0.01 to 50% by mass;
(9) A fuel oil composition comprising (X) a fuel oil and (Y) the additive for fuel oils as claimed in any one of Aspects (4) to (6); and
(10) A fuel oil composition as claimed in Aspect (9) wherein the content of the component (Y) is 0.01 to 1000 ppm by mass

The present invention provides a sulfur-based extreme-pressure additive used for lubricating oils and fuel oils exhibiting superior load-carrying capacity and wear resistance, and low corrosion attack against nonferrous metals and a lubricating oil composition and fuel oil composition containing the additive.

### BEST MODE FOR CARRYING OUT THE INVENTION

The compound which is used in the lubricating oil and fuel oil of the present invention is a disulfide compound having a structure represented by the general formula (I):

R¹OOC—CR³R⁴—CR⁵(COOR²)—S—S—CR¹⁰(COOR⁷)—CR⁸R⁹—COOR⁶ (I)

wherein R¹, R², R⁶, and R⁷ each independently represent a hydrocarbyl group having from 1 to 30 carbon atoms, preferably a hydrocarbyl group having from 1 to 20 carbon atoms, more preferably hydrocarbyl group having from 2 to 18 carbon atoms, and most preferably, hydrocarbyl group having from 3 to 18 carbon atoms. The hydrocarbyl group may be linear, branched, or cyclic and may contain an oxygen sulfur, or nitrogen atom. R¹, R², R⁶, and R⁷ may be the same or different and preferably should be the same for the production ease. R³ to R⁵ and R⁸ to R¹⁰ each independently represent a hydrogen atom or hydrocarbyl group having from 1 to 5 carbon atoms. Hydrogen, which can be readily available, is preferred.

It is preferred that the disulfide compound be produced by one of the following two processes, in the present invention. A first process involves oxidative coupling of a mercaptoalkanedicarboxylic acid diester, as a raw material, represented by the general formula (II) and/or general formula (III):

R¹OOC—CR³R⁴—CR⁵(COOR²)—SH (II)

R⁶OOC—CR⁸R⁹—CR¹⁰(COOR⁷)—SH (III)

wherein R¹ to R¹⁰ are as defined above.

The following products are prepared:

R¹OOC—CR³R⁴—CR⁵(COOR²)—S—S—CR¹⁰(COOR⁷)—CR⁸R⁹—COOR⁶,

R¹OOC—CR³R⁴—CR⁵(COOR²)—S—S—CR⁵(COOR²)—CR³R⁴—COOR¹, and

R⁶OOC—CR⁸R⁹—CR¹⁰(COOR⁷)—S—S—CR¹⁰(COOR⁷)—CR⁸R⁹—COOR⁶.

Examples of oxidizing reagent include oxygen, hydrogen peroxide, halogens i.e., iodine and bromine, hypohalous acids and salts thereof, sulfoxides i.e., dimethyl sulfoxide, and diisopropyl sulfoxide, and manganese (IV) oxide. Among these oxidizing agents preferably used are oxygen, hydrogen peroxide, and dimethyl sulfoxide, which are inexpensive and promotes production of the disulfide.

A second process of making the disulfide compound involves oxidative coupling of a mercaptoalkanedicarboxylic acid represented by the general formula (IV) and/or general formula (V):

HOOC—CR³R⁴—CR⁵(COOH)—SH (IV)

HOOC—CR⁸R⁹—CR¹⁰(COOH)—SH (V)

wherein R³ to R⁵ and are R⁸ to R¹⁰ are as defined above; and then esterifying the product with a monovalent alcohol having a hydrocarbyl group having from 1 to 30 carbon atoms and may contain an oxygen, sulfur, or nitrogen atom. Through the oxidative coupling, the following compounds are produced:

HOOC—CR³R⁴—CR⁵(—COOH)—S—S—CR¹⁰(COOH)—CR⁸R⁹—COOH,

HOOC—CR³R⁴—CR⁵(—COOH)—S—S—CR⁵(COOH)—CR³R⁴—COOH, and

HOOC—CR⁸R⁹—CR¹⁰(—COOH)—S—S—CR¹⁰(COOH)—CR⁸R⁹—COOH.

### Usable oxidizing agents are the same as above.

After the oxidative coupling, the product is esterified with an alcohol represented by the general formula (VI):

R¹¹—OH (VI)

wherein R¹¹ is as defined above. The esterification may be carried out by a normal process, namely, dehydrative condensation using an acid catalyst. Throughout the reactions the following compounds are produced:

R¹¹OOC—CR³R⁴—CR⁵(COOR¹¹)—S—S—CR¹⁰(COOR¹¹)—CR⁸R⁹—COOR¹¹,

R¹¹OOC—CR³R⁴—CR⁵(COOR¹¹)—S—S—CR⁵(COOR¹¹)—CR³R⁴—COOR¹¹, and

R¹¹OOC—CR⁸R⁹CR¹⁰(COOR¹¹)—S—S—CR¹⁰(COOR¹¹)—CR⁸R⁹—COOR¹¹.

Examples of the disulfide compounds represented by the general formula (I) include tetramethyl dithiomalate, tetraethyl dithiomalate, tetra-1-propyl dithiomalate, tetra-2-propyl dithiomalate, tetra-1-butyl dithiomalate tetra-2-butyl dithiomalate, tetraisobutyl dithiomalate, tetra-1-hexyl dithiomalate, tetra-1-octyl dithiomalate, tetra-1-(2-ethyl)hexyl dithiomalate, tetra-1-(3,5,5-trimethyl)hexyl dithiomalate, tetra-1-decyl dithiomalate, tetra-1-dodecyl dithiomalate, tetra-1-hexadecyl dithiomalate, tetra-1-octadecyl dithiomalate, tetrabenzyl dithiomalate, tetra-α-(methyl)benzyl dithiomalate, tetra-α,α-dimethylbenzyl dithiomalate, tetra-1-(2-methoxy)ethyl dithiomalate, tetra-1-(2-ethoxy)ethyl dithiomalate, tetra-1-(2-butoxy)ethyl dithiomalate, tetra-1-(2-ethoxy)ethyl dithiomalate, tetra-1-(2-butoxy)ethyl dithiomalate, and tetra-1-(2-phenoxy)ethyl dithiomalate.

These disulfide compounds exhibit superior load-carrying capacity and wear resistance as sulfur-based extreme pressure additives and can be used as additives for lubricating oils and fuel oils.

The additives for lubricating oils and fuel oils of the present invention may contain one or more disulfide compounds represented by the general formula (I)

The lubricating oil composition of the present invention comprises (A) a lubricating base oil and (B) the above-mentioned additive for lubricating oils. In the present invention examples of the lubricating oil composition include automotive lubricating oils used in drive equipment, such as internal combustion engines, automatic transmissions, dampers, and power steering systems, and gears; metalworking oils used in metal working such as cutting, grinding, and deformation processing; hydraulic oils being power transmission fluids used for power transmission, control, and buffering in hydraulic systems such as hydraulic equipment.

The lubricating base oil used as the component (A) in the lubricating oil composition of the present invention can be selected from mineral oils and synthetic oils, according to the applications and operation conditions of the composition without limitation. Examples of mineral oils include distillated oils prepared by atmospheric distillation or by vacuum distillation of residual oils of paraffinic crude oils, intermediate crude oils, or napthenic crude oils; and refined oils prepared by refining the distillated oils though an ordinary process, such as solvent-refined oils, hydro-refined oils, dewaxed oils, and white-clay-treated oils.

Examples of synthetic oils include low-molecular-weight polybutene, low-molecular-weight polypropylene, α-olefin having from 8 to 14 carbon atoms oligomers, and hydrogenated thereof, ester compounds such as polyol esters, e.g., fatty acid esters of trimethylolpropane and pentaerythritol, dibasic acid esters, aromatic polycarboxylate esters, and phosphate esters alkylaromatic compounds such as alkylbenzenes and alkylnaphthalenes, polyglycol oils such as polyalkylene glycol, and silicone oils.

These base oils may be used alone or in combination of two or more types.

The content of the component (B) being the additive for lubricating oils in the lubricating oil composition of the present invention is determined depending on its application and operation condition and generally ranges from 0.01 to 50% by mass. In the case of vehicle lubricating oils and hydraulic oils, the content ranges generally from 0.01 to 30% by mass, and preferably 0.01 to 10% by mass. In the case of metalworking oils, the content ranges generally from 0.1 to 60% by mass, and preferably 0.1 to 50% by mass, although the additive may be used alone.

The lubricating oil composition of the present invention may contain various additives, for example, other friction modifiers (oiliness agent and other extreme-pressure additive) and antiwear agents, ashless dispersants, metallic detergents, viscosity index improvers, pour-point depressant, rust inhibitors, metallic corrosion inhibitors, deforming agents, surfactants, and antioxidants, if necessary.

Examples of the other friction modifiers and antiwear agents include sulfide compounds such as olefin sulfides, dialkyl polysulfides, diarylalkyl polysulfides, and diaryl polysulfides; phosphorous compounds such as phosphate esters, thiophosphate esters, phosphite esters, alkyl hydrogen phosphites, amine salts of phosphate esters, and amine salts of phosphite esters; chlorinated compounds such as chlorinated fats and oils, chlorinated paraffins chlorinated fatty acid esters, and chlorinated fatty acids; esters such as alkyl or alkenyl maleate esters and alkyl or alkenyl succinate esters; organic acids such as alkyl or alkenyl maleic acids and alkyl or alkenyl succinic acids; and organometallic compounds such as naphthenate salts, zinc dithiophosphate (ZnDTP), zinc dithiocarbamate (ZnDTC), sulfurized oxymolybdenum organophosphorodithioate (MoDTP) and sulfurized oxymolybdenum dithiocarbamate (MoDTC).

Examples of the ashless dispersants include succinic imides, boron-containing succinic imides, benzylamines, boron-containing benzylamines, succinic esters, and amides of monovalent or divalent carboxylic acids such as fatty acids and succinic acid. Examples of the metallic detergents include neutral metal sulfonates, neutral metal phenates, neutral metal salicylates, neutral metal phosphonates, basic sulfonates, basic phenates, basic salicylates, basic phosphonates, perbasic sulfonates, perbasic phenates, perbasic salicylates, and perbasic phosphonates.

Examples of the viscosity index improvers include polymethacrylate, dispersion-type polymethacrylate, olefin copolymers such as ethylene-propylene copolymers, dispersion-type olefin copolymers, and styrene copolymers such as styrene-diene hydrogenated copolymers. Examples of the pour point depressants include polymethacrylate.

Examples of rust inhibitors include alkenylsuccinic acids and partial esters thereof. Examples of the metal corrosion inhibitors include benzotriazoles, benzimidazoles, benzothiazoles, and thiadiazoles. Examples of the deforming agents include dimethylpolysiloxane and polyacrylate. Examples or the surfactants include polyoxyethylene alkyl phenyl ether.

Examples of the antioxidants include amine antioxidants such as alkylated diphenylamines, phenyl-α-naphthylamine, and alkylated naphthylamines; phenolic antioxidants such as 2,6-di-t-butylcresol and 4,4′-methylenebis(2,6-di-t-butylphenol).

Examples of applications of the lubricating oil composition of the present invention include vehicle lubricating oils used in driving units, such as internal combustion engines, automatic transmissions, dampers, and power steering systems, and used in gears; metalworking oils used in metalworking such as cutting, grinding, and deformation processing; hydraulic oils being power transmission fluids used for power transmission, control, and buffering in hydraulic systems such as hydraulic equipment.

The fuel oil composition of the present invention includes (X) a fuel oil and (Y) the above-mentioned additive for fuel oils containing the disulfide compound.

Preferred examples of the fuel oils being the component (X) in the fuel oil composition of the present invention include highly hydro-refined fuel oils such as high-performance turbine fuel oils.

The content of the component (Y) being the additive for fuel oils in the fuel oil composition of the present invention resides in the range of, generally 0.01 to 1000 ppm by mass, and preferably 0.01 to 100 ppm by mass

The fuel oil composition of the present invention may contain various known additives, if necessary. Examples of such additives include antioxidants such as phenylenediamine diphenylamine, alkylphenol, and aminophenol antioxidants; detergents such as polyetheramine and polyalkylamine; metal deactivators such as Schiff compounds and thioamide compounds; surface ignition inhibitors such as organophosphorous compounds; deicing agents such as multivalent alcohols and ethers; combustion improvers such as alkali metal and alkaline earth metal salts of organic acids and sulfate esters of higher alcohols; antistatic agents such as anionic surfactants, cationic surfactants, and amphoteric surfactants; rust inhibitors such as alkenylsuccinate esters; oil markers such as quinizarin and coumarin; odorants such as natural essential oils and synthetic perfumes; and colorants such as azo dyes.

### EXAMPLES

The present invention will now be described in further detail by way of examples but should not be limited to these examples.

The friction coefficient wear scar width, and corrosiveness of the lubricating oil composition were determined by the following procedures:

### (1) Friction Coefficient and Wear Scar Width

Soda's Four-Ball Testing was carried out under the following conditions:

A hydraulic pressure load was gradually increased for 1080 seconds while each load [0.5, 0.7 0.9, 1.1, 3, or 1.5 kgf/cm² (×0.09807 MPa)] was maintained for 180 seconds at a number of rotations of 500 rpm and an oil temperature of 80° C. The friction coefficient was determined for each load, and the wear scar width was measured after the testing was completed.

### (2) Corrosiveness

According to JIS K-2513 “Petroleum Products—Corrosiveness to Copper—Copper Strip Test” at a testing temperature of 100° C. and a testing time of 3 hours and a test tube method, corrosiveness was measured. The tarnish of a copper strip was observed with reference to “Copper Strip Corrosion Standard”, and corrosiveness was evaluated as Subdivision Codes 1a to 4c. A smaller number in Subdivision Codes represents less corrosive and corrosiveness increases in alphabetical order.

### Preparative Example 1

### Production of tetra-1-octyl dithiomalate

Di-1-octyl thiomalate was oxidized with a dimethyl sulfoxide by the following procedure to produce tetra-1-octyl dithiomalate.

Into a 200 mL pear-shaped flask, 36.4 g of 1-octyl thiomalate and 39 g of dimethyl sulfoxide were placed the mixture was heated in an oil bath at 100° C. for 8 hours. Water and dimethyl sulfoxide were distilled out under reduced pressure. After cooling, the residue was dissolved in toluene, and was washed with an aqueous 5% sodium hydroxide solution and then with water. Toluene was distilled out under reduced pressure to recover 34.1 g of tetra-1-octyl dithiomalate.

### Preparative Example 2

### Production of tetra-1-(2-ethyl)hexyl dithiomalate

Tetra-1-(2-ethyl)hexyl dithiomalate was prepared as in Preparative Example 1 except that di-1-(2-ethyl)hexyl thiomalate was used instead of di-1-octyl thiomalate.

### Preparative Example 3

### Production of tetramethyl dithiomalate

Tetramethyl dithiomalate was prepared as in Preparative Example 1 except that distillation-purified dimethyl thiomalate was used instead of di-1-octyl thiomalate.

### Preparative Example 4

### Production of tetra-1-hexyl dithiomalate

Into a 200 mL pear-shaped flask, 30.0 g of tetramethyl dithiomalate which was prepared in Preparative Example 3, 69.2 g of 1-hexanol, and 1.2 g of p-toluenesulfonic acid monohydrate were placed and methanol was distilled out by spending 12 hours. After cooling, the residue was dissolved in toluene, and was washed with an aqueous 5% sodium hydroxide solution and then with water. Toluene and 1-hexanol were distilled out under reduced pressure to recover 108 g of tetra-1-hexyl dithiomalate.

### Preparative Example 5

### Production of tetra-1-(2-ethoxy)ethyl dithomalate

Tetra-1-(2-ethoxy)ethyl dithomalate was prepared as in Preparative Example 4 except that 2-ethoxyethanol was used instead of 1-butanol.

### Preparative Example 6

### Production of tetra-1-butyl dithiomalate

Into a 2 L four-necked flask with a stirring device, 200 g of thiomalic acid and 900 mL of water were added, and 61.0 g of 35% hydrogen peroxide solution was gradually added by spending 2 hours with stirring at a temperature of 25° C. to 35° C. The temperature was raised to 60° C. and the mixture was stirred for 1 hour. The solution was transferred to a 2 L pear-shaped flask and water was distilled out under reduced pressure. To the residue, 600 mL of toluene, 237 g of 1-butanol and 8 g of p-toluenesulfonic acid monohydrate were added. After a Dean-Stark trap was attached, the solution was heated and refluxed for 10 hours. After cooling, the product was washed with an aqueous 5% sodium hydroxide solution and then water. Toluene and 1-butanol were distilled out under reduced pressure to recover 317 g of tetra-t-butyl dithiomalate.

### Preparative Example 7

### Production of tetra-1-hexyl dithiomalate

Tetra-1-hexyl dithiomalate was prepared as in Preparative Example 6 except that 1-hexanol was used instead of 1-butanol

### Preparative Example 8

### Production of tetra-1-octyl dithiomalate

Tetra-1-octyl dithiomalate was prepared as in Preparative Example 6 except that 1-octanol was used instead of 1-butanol.

### Example 1

Tetra-1-octyl dithiomalate produced in Preparative Example 1 was added to mineral oil of a 500 neutral fraction (P500N) such that the additive content in the composition was 1% by mass to prepare a lubricating oil composition The observed properties of the composition are shown in Table 1.

### Examples 2 to 7

As shown in Table 1, the additives prepared in Preparative Examples 2 and 4 to 8 were each added mineral oil of a 500 neutral fraction (HG500) such that the additive content in the composition was 1% by mass to prepare lubricating oil compositions. The observed properties of the compositions are shown in Table 1.

### Comparative Example 1

Mineral oil of a 500 neutral fraction (P500N) was evaluated as in Example 1 with addition of no additive. The results are shown in Table 1.

TABLE 1

| | | | | | | | | | | Comparative |
|---|---|---|---|---|---|---|---|---|---|---|
| | | | Example 1 | Example 2 | Example 3 | Example 4 | Example 5 | Example 6 | Example 7 | Example 1 |
| Preparation of additive | | | Preparative | Preparative | Preparative | Preparative | Preparative | Preparative | Preparative | — |
| | | | Example 1 | Example 2 | Example 4 | Example 5 | Example 6 | Example 7 | Example 8 | |
| Friction | Load × 98.07 | 0.5 | 0.031 | 0.044 | 0.042 | 0.034 | 0.044 | 0.042 | 0.038 | 0.044 |
| Coefficient | (kPa) | 0.7 | 0.048 | 0.055 | 0.054 | 0.049 | 0.055 | 0.055 | 0.051 | 0.055 |
| | | 0.9 | 0.053 | 0.064 | 0.059 | 0.054 | 0.063 | 0.061 | 0.059 | 0.064 |
| | | 1.1 | 0.055 | 0.065 | 0.062 | 0.057 | 0.065 | 0.065 | 0.059 | 0.070 |
| | | 1.3 | 0.057 | 0.069 | 0.063 | 0.058 | 0.066 | 0.067 | 0.060 | 0.072 |
| | | 1.5 | 0.058 | 0.072 | 0.066 | 0.061 | 0.066 | 0.069 | 0.061 | 0.076 |

| | | | | | | | | |
|---|---|---|---|---|---|---|---|---|
| Wear scar width (mm) | 0.41 | 0.47 | 0.41 | 0.36 | 0.36 | 0.42 | 0.42 | 0.52 |
| Corrosiveness to copper strip | 1b | 1b | 1b | 1b | 1b | 1b | 1b | 1b |

Examples and Comparative Example show that the lubricating oil compositions containing the additives of the present invention each has a low friction coefficient and a small wear scar width and thus high load-bearing capacity and wear resistance.

### INDUSTRIAL APPLICABILITY

The additive for lubricating oils and fuel oils and the lubricating oil composition and fuel oil composition containing the additive of the present invention show superior load-bearing capacity and wear resistance and are useful in the fields of a variety of lubricating oils and fuel oils.

1. An additive for lubricating oils comprising a disulfide compound, as the main component, represented by the general formula (I).
R¹OOC—CR³R⁴—CR⁵(COOR²)—S—S—CR¹⁰(CCOR⁷)—CR⁸R⁹—COOR⁶ (I)
wherein R¹, R², R⁶, and R⁷ each independently represent a hydrocarbyl group having from 1 to 30 carbon atoms and may contain an oxygen, sulfur, or nitrogen atom; and R³ to R⁵ and R⁸ to R¹⁰ each independently represent a hydrogen atom, or hydrocarbyl group having from 1 to 5 carbon atoms.

2. An additive for lubricating oils comprising a disulfide compound, as the main component, prepared by oxidative coupling of a mercaptoalkanedicarboxylic acid diester represented by the general formula (II) and/or general formula (III)
R¹OOC—CR³R⁴—CR⁵(COOR²)—SH (II)
wherein R¹ and R² represent a hydrocarbyl group having from 1 to 30 carbon atoms and may contain an oxygen, sulfur, or nitrogen atom; and R³ to R⁵ each independently represent a hydrogen atom or hydrocarbyl group having from 1 to 5 carbon atoms:
R⁶OOC—CR⁸R⁹—CR¹⁰(COOR⁷)—SH (III)
where R⁶ and R⁷ represent a hydrocarbyl group having from 1 to 30 carbon atoms and may contain an oxygen, sulfur, or nitrogen atom; and R⁸ to R¹⁰ each independently represent a hydrogen atom or hydrocarbyl group having from 1 to 5 carbon atoms.

3. An additive for lubricating oils comprising a disulfide compound, as the main component, prepared by oxidative coupling of a mercaptoalkanedicarboxylic acid represented by the general formula (IV) and/or general formula (V):
HOOC—CR³R⁴—CR⁵(COOH)—SH (IV)
wherein R³ to R⁵ each independently represent a hydrogen atom or a hydrocarbyl group having from 1 to 5 carbon atoms:
HOOC—CR⁸R⁹—CR¹⁰(COOH)—SH (V)
wherein R⁸ to R¹⁰ each independently represent a hydrogen atom or a hydrocarbyl group having from 1 to 5 carbon atoms; and then esterifying the product with an alcohol represented by the general formula (VI):
R¹¹—OH (VI)
wherein R¹¹ represents a hydrocarbyl group having from 1 to 30 carbon atoms and may contain an oxygen sulfur, or nitrogen atom.

4. An additive for fuel oils comprising a disulfide compound, as the main component represented by the general formula (I):
R¹OOC—CR³R⁴—CR⁵(COOR²)—S—S—CR¹⁰(COOR⁷)—CR⁸R⁹—COOR⁶ (I)
wherein R¹, R², R⁶, and R⁷ each independently represent a hydrocarbyl group having from 1 to 30 carbon atoms and may contain an oxygen, sulfur, or nitrogen atom; and R³ to R⁵ and R⁸ to R¹⁰ each independently represent a hydrogen atom or hydrocarbyl group having from 1 to 5 carbon atoms.

5. An additive for fuel oils comprising a disulfide compound as the main component, prepared by oxidative coupling of a mercaptoalkanedicarboxylic acid diester represented by the general formula (II) and/or general formula (III):
R¹OOC—CR³R⁴—CR⁵(COOR²)—SH (II)
wherein R¹ and R² represent a hydrocarbyl group having from 1 to 30 carbon atoms and may contain an oxygen, sulfur, or nitrogen atom; and R³ to R⁵ each independently represent a hydrogen atom or hydrocarbyl group having from 1 to 5 carbon atoms:
R⁶OOC—CR⁸R⁹—CR¹⁰(COOR⁷)—SH (III)
wherein R⁶ and R⁷ represent a hydrocarbyl group having from 1 to 30 carbon atoms and may contain an oxygen, sulfur, or nitrogen atom; and R⁶ to R¹⁰ each independently represent a hydrogen atom or hydrocarbyl group having from 1 to 5 carbon atoms.

6. An additive for fuel oils comprising a disulfide compound, as the main component, prepared by oxidative coupling of a mercaptoalkanedicarboxylic acid represented by the general formula (IV) and/or general formula (V):
HOOC—CR³R⁴—CR⁵(COOH)—SH (IV)
wherein R³ to R⁵ each independently represent a hydrogen atom or a hydrocarbyl group having from 1 to 5 carbon atoms:
HOOC—CR⁸R⁹—CR¹⁰(COOH)—SH (V)
wherein R⁸ to R¹⁰ each independently represent a hydrogen atom or a hydrocarbyl group having from 1 to 5 carbon atoms; and then esterifying the product with an alcohol represented by the general formula (VI):
R¹¹—OH (VI)
wherein R¹¹ represents a hydrocarbyl group having from 1 to 30 carbon atoms and may contain an oxygen, sulfur, or nitrogen atom.

7. A lubricating oil composition comprising (A) a lubricating base oil and (B) the additive for lubricating oils as claimed in any one of claims 1 to 3.

8. A lubricating oil composition as claimed in claim 7 wherein the content of the component (B) is 0.01 to 50% by mass.

9. A fuel oil composition comprising (X) a fuel oil and (Y), the additive for fuel oils as claimed in any one of claims 4 to 6.

10. A fuel oil composition as claimed in claim 9 wherein the content of the component (Y) is 0.01 to 1000 ppm by mass.
